(19) United States
(12) Patent Application Publication
(10) Pub. No.: US 2025/0339629 A1
(43) Pub. Date: Nov. 6, 2025

(54) NEBULIZER AND HOUSING ASSEMBLY THEREOF

(71) Applicant: HCMed Innovations Co., LTD., Taipei City (TW)

(72) Inventors: HUNG-WEN CHEN, New Taipei City (TW); CHUN-CHIA JUAN, New Taipei City (TW)

(21) Appl. No.: 19/193,937

(22) Filed: Apr. 29, 2025

Related U.S. Application Data
(60) Provisional application No. 63/641,931, filed on May 2, 2024.

(30) Foreign Application Priority Data
Apr. 28, 2025 (CN) 202510544441.6

(51) Int. Cl. A61M 11/02

(52) U.S. Cl. CPC A61M 11/02

(57) ABSTRACT

A nebulizer includes a main body, a housing assembly, a cup body, and a nebulizing module. The housing assembly includes a housing and a flexible shielding member. The housing is engaged with the main body. An air chamber is provided inside the housing. The housing includes a mouthpiece. A sidewall of the housing is provided with a plurality of openings, and the plurality of openings are in fluid communication with the mouthpiece via the air chamber. The flexible shielding member covers at least one of the openings. The cup body is engaged with the housing. A liquid storage chamber is provided inside the cup body. A bottom of the housing is provided with a through hole that is in fluid communication with the liquid storage chamber, and the liquid storage chamber is in fluid communication with the air chamber via the through hole.

### CROSS-REFERENCE TO RELATED PATENT APPLICATION

This application claims the benefit of priority to China Patent Application No. 202510544441.6, filed on Apr. 28, 2025, in the People's Republic of China. The entire content of the above identified application is incorporated herein by reference.

This application claims the benefit of priority to the U.S. Provisional Patent Application Ser. No. 63/641,931, filed on May 2, 2024, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a nebulizer and a housing assembly thereof, and more particularly to a nebulizer and a housing assembly thereof capable of improving the breathing pattern of the user.

### BACKGROUND OF THE DISCLOSURE

In general, when a user intends to increase the deposition of aerosolized drug particles in their lungs during the operation of a nebulizer to enhance therapeutic efficacy, in addition to adjusting the mass median aerodynamic diameter (MMAD) of the aerosolized particles to control deposition in specific regions of the lungs, the user can also breathe deeply and slowly to further improve the deposition ratio of the aerosolized particles within their lungs.

To guide the user in maintaining stable and slow breathing during nebulization therapy, it is common to reduce the size of an air inlet of the nebulizer so that airflow resistance during inhalation can be increased. However, while such a design can help slow down the user's inhalation rate, it also increases the airflow resistance during exhalation, causing discomfort and affecting the user's tolerance to prolonged treatment.

Therefore, how to overcome the above-mentioned problem through an improvement in structural design of the airflow passage inside the nebulizer has become an important issue to be addressed in the related art.

### SUMMARY OF THE DISCLOSURE

Based on the above-referenced technical inadequacy, the present disclosure provides a nebulizer and a housing assembly, so as to address an issue of existing nebulizers being unable to improve therapeutic efficacy while minimizing the impact on the user's breathing comfort.

In order to solve the above-mentioned problems, one of the technical aspects adopted by the present disclosure is to provide a nebulizer, which includes a main body, a housing assembly, a cup body, and a nebulizing module. The housing assembly includes a housing and a flexible shielding member. The housing is engaged with the main body. An air chamber is provided inside the housing. The housing includes a mouthpiece. A sidewall of the housing is provided with a plurality of openings, and the plurality of openings are in fluid communication with the mouthpiece via the air chamber. The flexible shielding member is disposed on the sidewall of the housing. The flexible shielding member covers at least one of the openings. The flexible shielding member is configured to dynamically adjust an airflow passing through the openings based on a direction of the airflow entering or exiting the mouthpiece. The cup body is engaged with the housing. A liquid storage chamber is provided inside the cup body. A bottom of the housing is provided with a through hole that is in fluid communication with the liquid storage chamber, and the liquid storage chamber is in fluid communication with the air chamber via the through hole. The nebulizing module is disposed in the cup body and located directly above the through hole.

In order to solve the above-mentioned problems, another one of the technical aspects adopted by the present disclosure is to provide a housing assembly, which includes a housing and a flexible shielding member. The housing has an air chamber inside. The housing includes a mouthpiece. A sidewall of the housing is provided with a plurality of openings, and the plurality of openings are in fluid communication with the mouthpiece via the air chamber. The flexible shielding member is disposed on the sidewall of the housing. The flexible shielding member covers an outer end of at least one of the openings. The flexible shielding member is configured to dynamically adjust an airflow passing through the openings based on a direction of the airflow entering or exiting the mouthpiece.

Therefore, in the nebulizer and the housing thereof provided by the present disclosure, the flexible shielding member is disposed on the outer side of the at least one of the openings, so as to cover or uncover the at least one of the openings based on an airflow direction through the mouthpiece, thereby dynamically adjusting an airflow volume passing through the openings. Accordingly, when the user inhales through the nebulizer, the airflow direction causes the flexible shielding member to partially cover the openings, increasing an inhalation resistance and guiding the user to inhale a nebulized medication slowly and stably. On the other hand, when the user exhales through the nebulizer, the airflow direction keeps the flexible shielding member open without covering the openings, thereby avoiding increased exhalation resistance and ensuring breathing comfort. Accordingly, therapeutic efficacy of the user can be enhanced, and discomfort during inhalation and exhalation can be reduced, thereby improving the treatment experience for the user.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a first schematic view of a nebulizer according to a first embodiment of the present disclosure;

FIG. 2 is a second schematic view of the nebulizer according to the first embodiment of the present disclosure;

FIG. 3 is a schematic exploded view of the nebulizer according to the first embodiment of the present disclosure;

FIG. 4 is a schematic cross-sectional view of the nebulizer according to the first embodiment of the present disclosure;

FIG. 5 is a first schematic view of a housing assembly according to the first embodiment of the present disclosure;

FIG. 6 is a second schematic view of the housing assembly according to the first embodiment of the present disclosure;

FIG. 7 is a schematic view of a flexible shielding member of the housing assembly according to the first embodiment of the present disclosure;

FIG. 8 is a schematic cross-sectional view of the housing assembly during the user's inhalation according to the first embodiment of the present disclosure;

FIG. 9 is a schematic cross-sectional view of the housing assembly during the user's exhalation according to the first embodiment of the present disclosure;

FIG. 10 is a first schematic view of the housing assembly according to a second embodiment of the present disclosure;

FIG. 11 is a schematic exploded view of the housing assembly according to the second embodiment of the present disclosure;

FIG. 12 is a second schematic view of the housing assembly according to the second embodiment of the present disclosure;

FIG. 13 is a schematic view of a flexible shielding member of the housing assembly according to the second embodiment of the present disclosure;

FIG. 14 is a schematic cross-sectional view of the housing assembly during the user's inhalation according to the second embodiment of the present disclosure;

FIG. 15 is a schematic cross-sectional view of the housing assembly during the user's exhalation according to the second embodiment of the present disclosure;

FIG. 16 is a schematic exploded view of another implementation of the housing assembly and the flexible shielding member according to the second embodiment of the present disclosure;

FIG. 17 is a partial schematic exploded view of the housing assembly according to a third embodiment of the present disclosure;

FIG. 18 is a schematic view of a nebulizer according to the third embodiment of the present disclosure;

FIG. 19 is a schematic cross-sectional view of the housing assembly during the user's inhalation according to the third embodiment of the present disclosure; and

FIG. 20 is a schematic cross-sectional view of the housing assembly during the user's exhalation according to the third embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

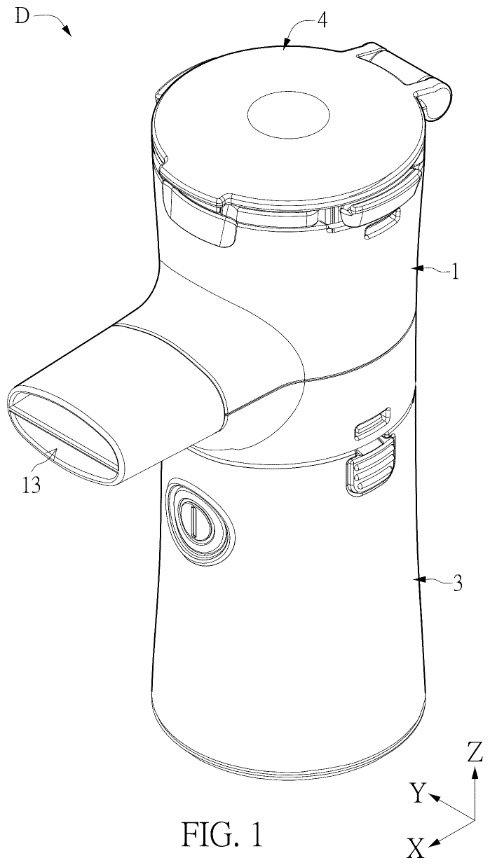

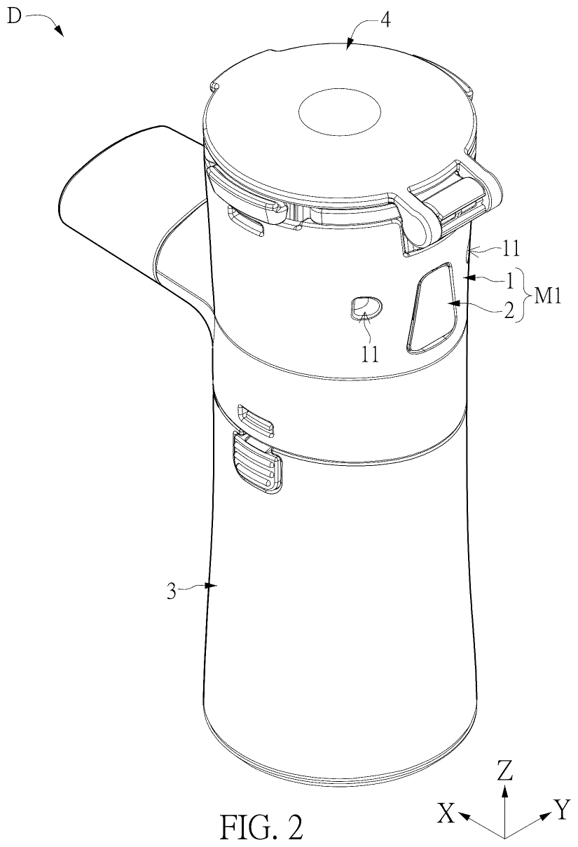

Referring to FIG. 1 to FIG. 3, a first embodiment of the present disclosure provides a nebulizer D, which includes a housing assembly M1, a main body 3, and a cup body 4. The housing assembly M1 includes a housing 1 and a flexible shielding member 2.

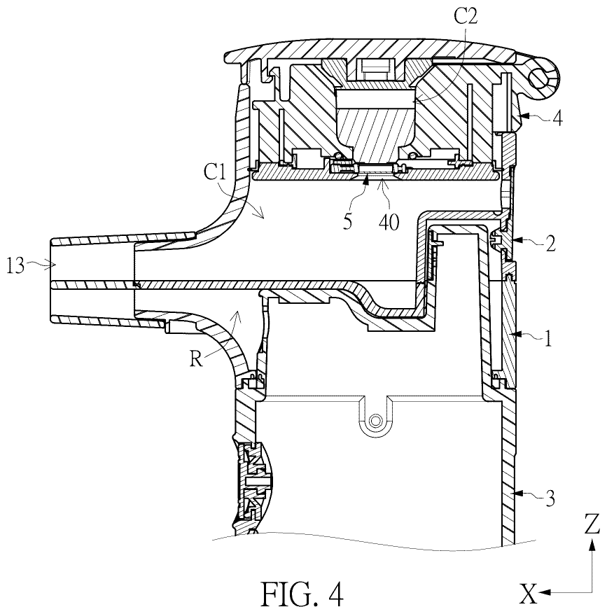

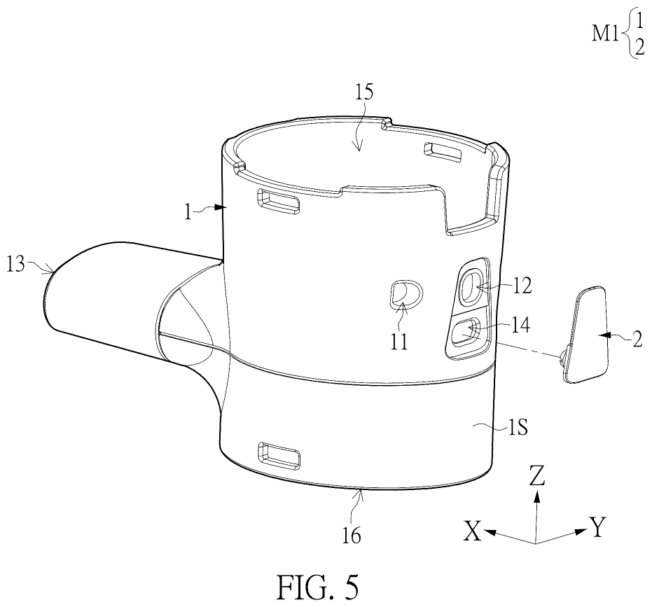

Reference is made to FIGS. 4 and 5. The housing 1 includes a mouthpiece 13, an upper port 15, and a lower port 16. The cup body 4 is engaged with the housing 1 via the upper port 15, and the main body 3 is engaged with the housing 1 via the lower port 16. The housing 1 has a hollow space inside to define an air chamber C1. A sidewall 1S of the housing 1 is provided with a plurality of openings, and the openings are in fluid communication with the mouthpiece 13 via the air chamber C1.

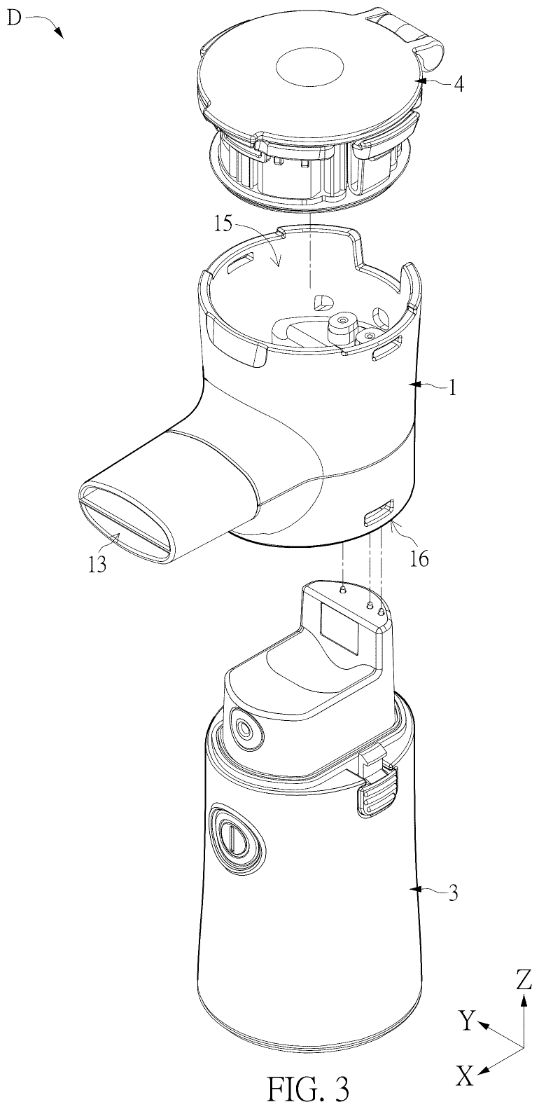

Referring to FIGS. 3 to 5, the openings are further divided into first openings 11 and second openings 12. Quantities of the first openings 11 and the second openings 12 are not limited in the present disclosure. In the first embodiment, two first openings 11 and one second opening 12 are provided, and the second opening 12 is located between the two first openings 11. Furthermore, the openings (i.e., the two first openings 11 and one second opening 12) and the mouthpiece 13 are located on opposite sides of the housing 1. The first openings 11 and the second openings 12 serve as inlets and outlets to ensure airflow circulation within the air chamber C1.

The cup body 4 defines a liquid storage chamber C2 inside for storing liquid medication (not shown in the figures). A bottom of the cup body 4 is provided with a through hole 40 that is in fluid communication with the liquid storage chamber C2. The liquid storage chamber C2 is in fluid communication with the air chamber C1 via the through hole 40. The nebulizer D further includes a nebulizing module 5. The nebulizing module 5 is disposed inside the cup body 4 and positioned directly above the through hole 40. For example, the nebulizing module 5 can be made of piezoelectric ceramic material and capable of generating high-frequency vibrations under an appropriate driving voltage to nebulize the liquid medication into an aerosol (not shown in the figures). After the liquid medication passes through the through hole 40, the liquid medication is nebulized to form the aerosol by the nebulizing module 5 and stored in the air chamber C1. Subsequently, the aerosol is inhaled by a user (i.e., a patient, not shown in the figures) through the mouthpiece 13 into the upper respiratory tract and lungs.

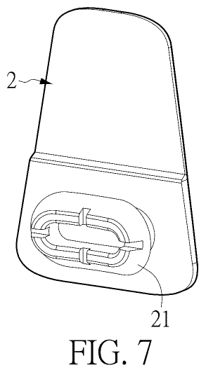

Referring to FIGS. 5 to 7, the flexible shielding member 2 is disposed on the sidewall 1S of the housing 1 and covers the second opening 12. Specifically, the sidewall 1S of the housing 1 is further provided with a fastening hole 14, and the flexible shielding member 2 includes a fastening member 21. The shape and the position of the fastening member 21 correspond to those of the fastening hole 14. The flexible shielding member 2 is fixedly connected to the sidewall 1S by the fastening member 21 being engaged with the fastening hole 14. It should be noted that a quantity of the fastening member 21 and the fastening holes 14 is not limited in the present disclosure. For example, the flexible shielding member 2 can be made of silicone, thermoplastic elastomer (TPE), polyurethane (PU), or other similar materials, but the present disclosure is not limited thereto.

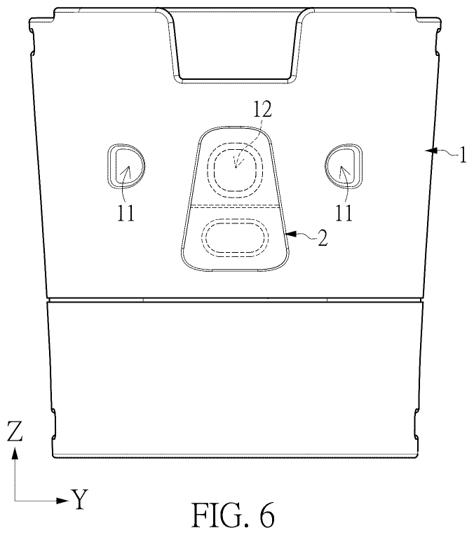

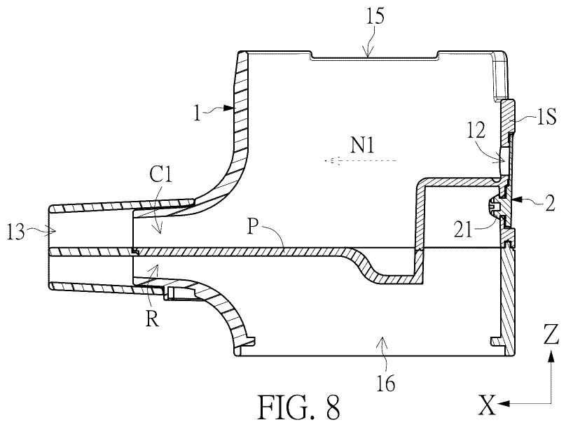

In the first embodiment, the flexible shielding member 2 is a sheet-shaped structure. As shown in FIGS. 5 and 6, a projection of the flexible shielding member 2 that is projected onto the sidewall 1S of the housing 1 completely overlaps the second opening 12, and the flexible shielding member 2 covers an outer end of the second opening 12. In other words, the second opening 12 is completely covered by the projection of the flexible shielding member 2 on the sidewall 1S of the housing 1 (i.e., a projected area of the flexible shielding member 2 is equal to or larger than an aperture size of the second opening 12). Therefore, referring to FIG. 4 and FIG. 8, when the patient inhales through the mouthpiece 13 and draws the aerosol in the air chamber C1 into the body, the airflow within the air chamber C1 flows toward the mouthpiece 13 (i.e., an airflow direction N1 in FIG. 8), thereby pulling the flexible shielding member 2 to tightly adhere to the second opening 12.

Since the second opening 12 is completely covered by the flexible shielding member 2, and the flexible shielding member 2 is tightly adhered to the second opening 12, the air in the external environment is prevented from entering the air chamber C1 through the second opening 12, thereby increasing the inhalation resistance during the patient's inhalation. In order to smoothly inhale the aerosol (i.e., the nebulized medication), the patient will naturally adjust their breathing rhythm to adopt a slower and deeper inhalation. The slower and deeper inhalation not only facilitates the smooth entry of the aerosol into the respiratory tract, but also allows the aerosol to penetrate deeper into the lungs, thereby enabling the drug particles to be effectively deposited in the lung tissues and enhancing the therapeutic efficacy. Additionally, the proper inhalation resistance helps the patient inhale stably, avoiding excessively rapid inhalation that causes premature deposition of the drug particles in the mouth or throat, thereby improving drug delivery efficiency.

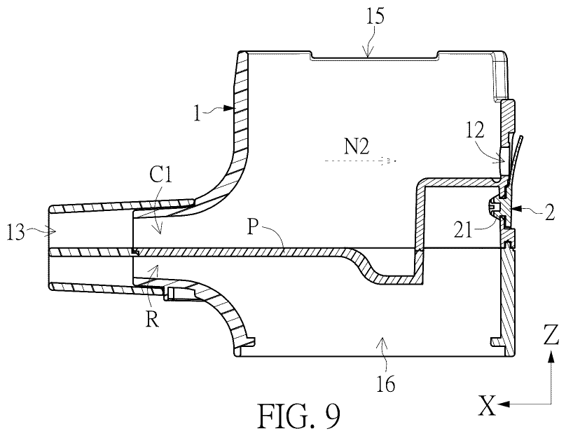

On the other hand, referring to FIG. 4 and FIG. 9, when the patient exhales through the mouthpiece 13, the airflow flows toward the first openings 11 and the second opening 12 on the housing 1 (i.e., an airflow direction N2 in FIG. 9). Since the flexible shielding member 2 is flexible and sheet-shaped, it lifts when pushed by the airflow, such that the second opening 12 is exposed on the housing 1 and the exhaled air can exit smoothly. In other words, during the patient's exhalation, the flexible shielding member 2 does not cover the second opening 12, effectively reducing the exhalation resistance and ensuring smooth breathing to improve the patient's comfort. In addition, the design of the nebulizer D prevents pressure buildup inside the housing 1 during exhalation, avoiding any negative impact on the nebulization performance in subsequent inhalation, thereby improving the stability and efficiency of the nebulizer D. In other words, the flexible shielding member 2 dynamically adjusts a volume of the airflow passing through the openings based on a direction of the airflow entering or exiting the mouthpiece 13, and the function of the flexible shielding member 2 is equivalent to a one-way valve that permits unidirectional airflow.

### Second Embodiment

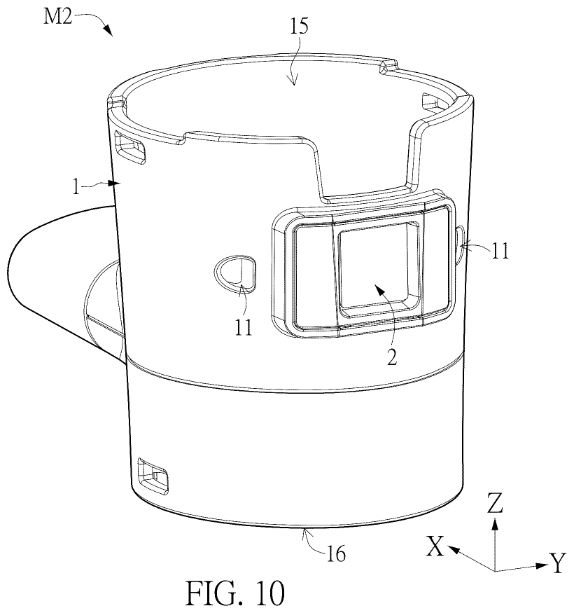

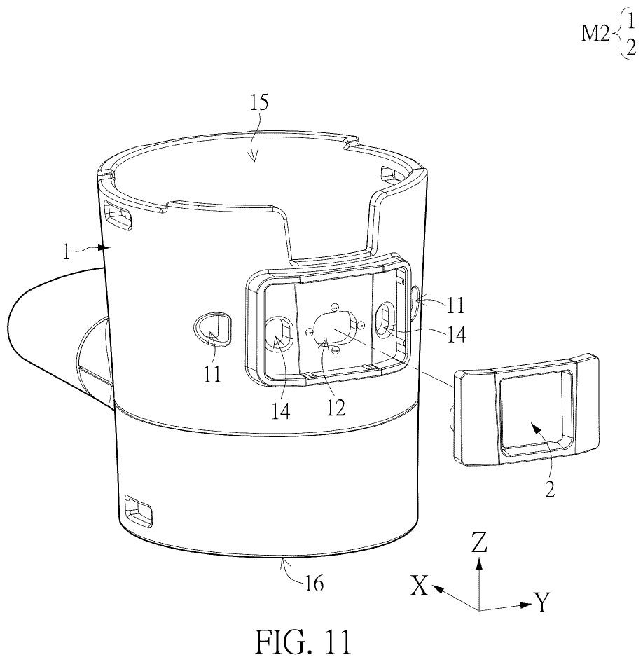

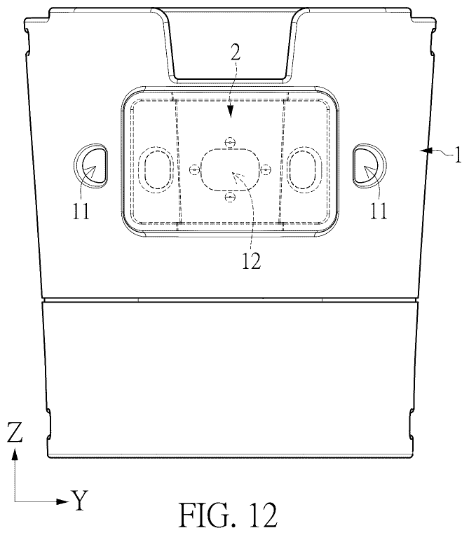

Referring to FIGS. 10 to 13, a second embodiment of the present disclosure provides a housing assembly M2. The structure of the housing assembly M2 in the second embodiment is similar to that of the housing assembly M1 in the first embodiment, and the similarities will not be reiterated herein. The main difference resides in that the structural configuration of the flexible shielding member 2 in the second embodiment is different from that in the first embodiment. Therefore, the housing assembly M2 of the second embodiment is also applicable to the nebulizer D described in the first embodiment (shown in FIGS. 1 to 4). That is, the housing assembly M1 in the first embodiment can be replaced with the housing assembly M2 of the second

The housing assembly M2 includes a housing 1 and a flexible shielding member 2. A sidewall 1S of the housing 1 is provided with two first openings 11 and one second opening 12, and the second opening 12 is located between the two first openings 11. The flexible shielding member 2 covers the second opening 12. Furthermore, in the second embodiment, the sidewall 1S of the housing 1 is further provided with two fastening holes 14, and the second opening 12 is located between the two fastening holes 14. The flexible shielding member 2 includes two fastening members 21 that correspond to the two fastening holes 14. The flexible shielding member 2 is fixedly connected to the housing 1 by the two fastening members 21 being engaged respectively with the two fastening holes 14. In addition to the two fastening members 21, the flexible shielding member 2 further includes a hollow frame 22 and a shielding sheet 23. The shielding sheet 23 is flexible and is formed into a thin sheet. The two fastening members 21 are disposed on the hollow frame 22. A first end 231 at the upper side of the shielding sheet 23 is connected to the hollow frame 22, while a second end 232 at the lower side of the shielding sheet 23 is a free end.

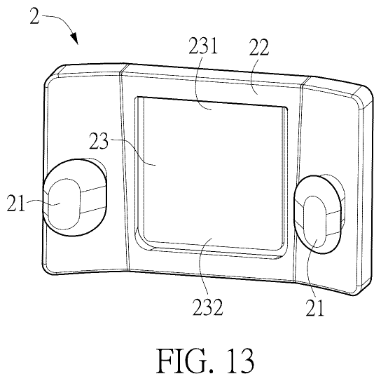

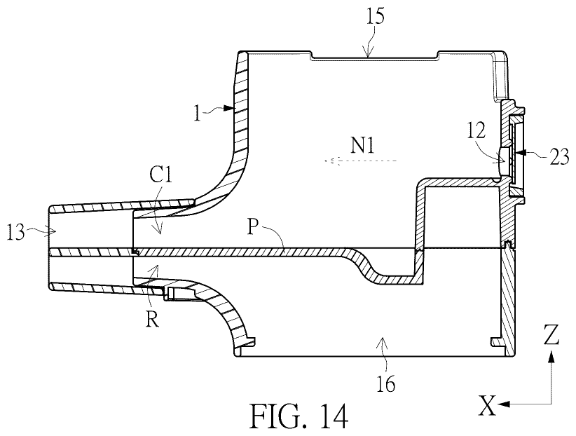

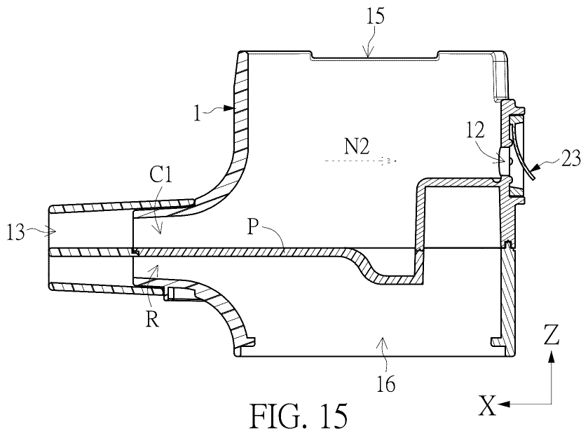

Reference is made to FIGS. 13 to 15. When the patient inhales through the mouthpiece 13 and draws the aerosol in the air chamber C1 into the body, the airflow within the air chamber C1 flows toward the mouthpiece 13 (i.e., an airflow direction N1 in FIG. 14), and the shielding sheet 23 of the flexible shielding member 2 is pulled to tightly adhere to the second opening 12. As a result, the air in the external environment is prevented from entering the air chamber C1 through the second opening 12, causing flow resistance to increase and guiding the patient to adjust their breathing rhythm by adopting a slower and deeper inhalation. On the other hand, when the patient exhales through the mouthpiece 13, the airflow flows toward the first openings 11 and the second opening 12 (i.e., an airflow direction N2 in FIG. 15), and the shielding sheet 23 of the flexible shielding member 2 is lifted by the airflow, such that the second opening 12 is exposed on the housing 1 and the exhaled air can exit smoothly, thereby effectively reducing exhalation resistance and ensuring smooth breathing.

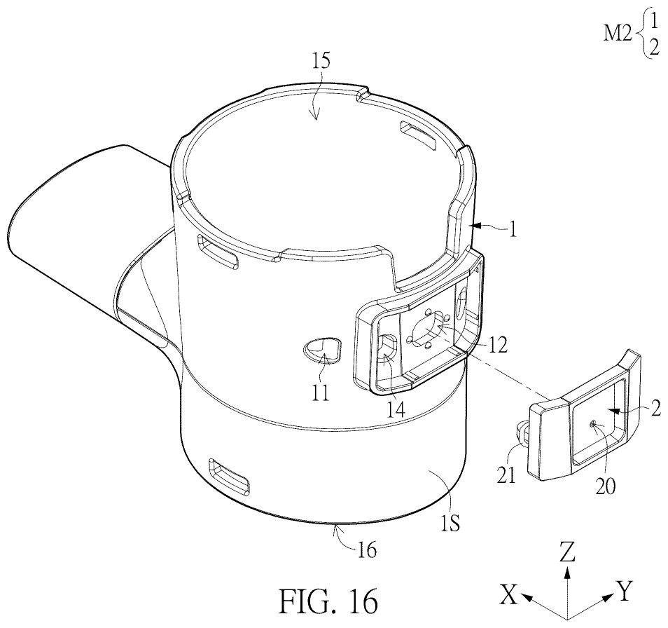

Furthermore, reference is made to FIG. 16. In order to further promote airflow circulation between the inside and outside of the housing 1, the flexible shielding member 2 is provided with at least one vent hole 20 on the shielding sheet 23. The second opening 12 is in fluid communication with the vent hole 20. When the patient inhales, a guiding airflow can be generated at the center of the air chamber C1 through the vent hole 20, thereby enhancing the aerosol guiding effect within the air chamber C1. It should be noted that the aperture size of the vent hole 20 is significantly smaller than that of the first openings 11 and the second opening 12, such that the flexible shielding member 2 can keep covering the second opening 12 to increase the inhalation resistance.

### Third Embodiment

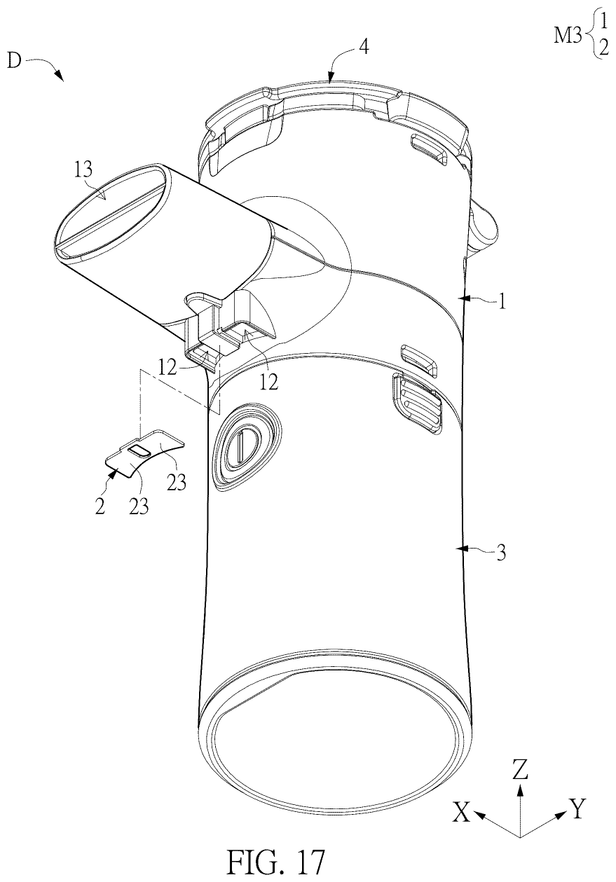

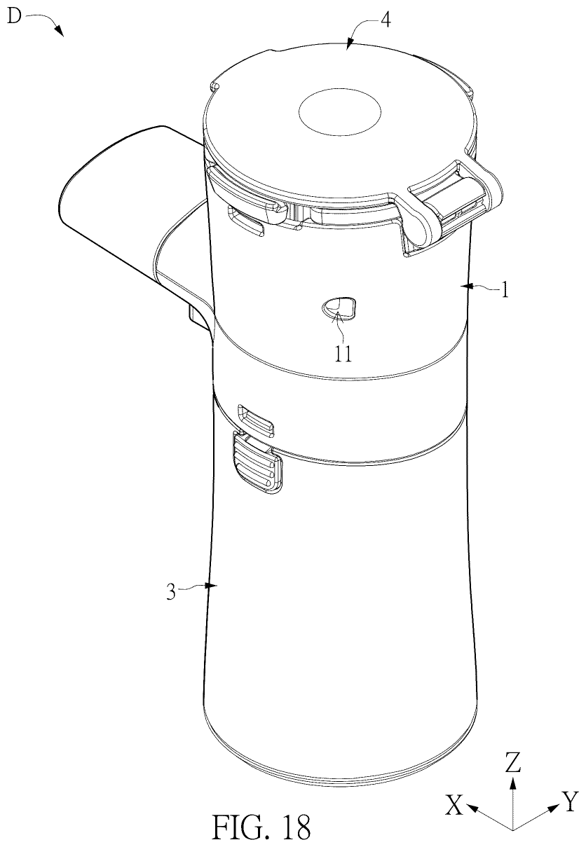

Referring to FIGS. 17 and 18, a third embodiment of the present disclosure provides a housing assembly M3 applicable to the nebulizer D. The structure of the housing assembly M3 in the third embodiment, including a housing 1 and a flexible shielding member 2, is similar to that of the housing assembly M1 in the first embodiment, and the similarities will not be reiterated herein. The main difference resides in that the structural configuration of the housing assembly M3 of the third embodiment is different from that of the first

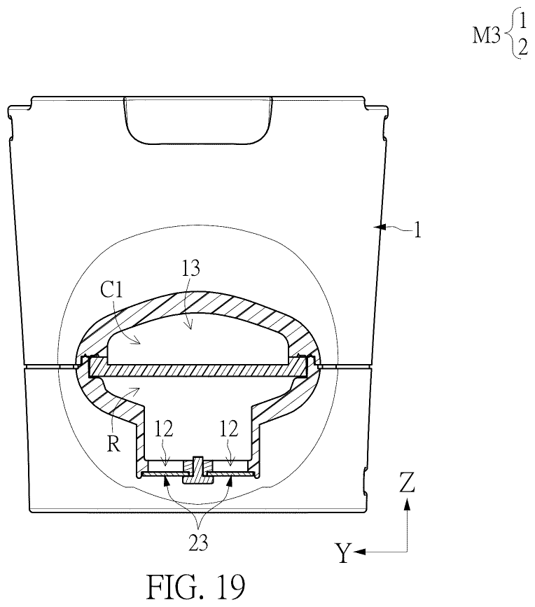

As shown in FIGS. 17 and 18, the housing 1 includes two first openings 11 and two second openings 12. The opening directions of the first openings 11 is different from those of the second openings 12. The two first openings 11 are disposed on a sidewall 1S of the housing 1, and the two first openings 11 and the mouthpiece 13 are located on opposite sides of the housing 1. Referring to FIGS. 4 and 19, the housing 1 further includes a partition wall P. The partition wall P is disposed within the mouthpiece 13 and extends backward to isolate the upper port 15 and the lower port 16. A hollow space above the partition wall P forms an air chamber C1, while another hollow space beneath the partition wall P forms a sensing chamber R. The sensing chamber R is configured to accommodate a sensing component (not shown in the figures). For example, the sensing component can be a pressure sensor. The air chamber C1 and the sensing chamber R are spatially isolated and not in fluid communication with each other. The two second openings 12 are in fluid communication with the sensing chamber R and disposed beneath the mouthpiece 13 of the housing 1. Accordingly, in the third embodiment, the second openings 12 are in fluid communication with the sensing chamber R that is located beneath the partition wall P, and the two second openings 12 are positioned closer to the mouthpiece 13 than the first openings 11. The flexible shielding member 2 includes two shielding sheets 23 that respectively cover the two second openings 12. In other words, the housing assembly M3 of the third embodiment includes a plurality of one-way valves. In addition, each of the housing assemblies M1 and M2 of the first and second embodiments includes a single one-way valve.

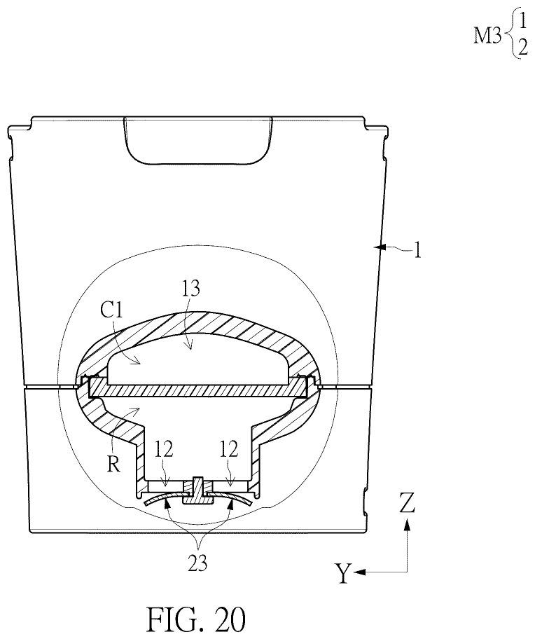

Reference is made to FIGS. 17, 19, and 20. When the patient inhales through the mouthpiece 13, the airflow within both the air chamber C1 and the sensing chamber R flows toward the mouthpiece 13. At this time, the two shielding sheets 23 located beneath the sensing chamber R are pulled by the airflow and tightly adhere to the second openings 12, preventing the air in the external environment from entering the sensing chamber R through the second openings 12, thereby increasing the flow resistance and guiding the patient to adjust their breathing rhythm and adopt a slower and deeper inhalation pattern. On the other hand, when the patient exhales through the mouthpiece 13, the airflow flows toward the first openings 11 of the air chamber C1 and the second openings 12 of the sensing chamber R. At this time, the two shielding sheets 23 located beneath the sensing chamber R are pushed and lifted by the airflow, such that the second openings 12 are exposed on the housing 1 and the exhaled air can exit smoothly, thereby effectively reducing exhalation resistance and ensuring smooth breathing.

### Beneficial Effects of the Embodiments

Existing nebulizers are unable to simultaneously improve therapeutic efficacy and maintain the patient's breathing comfort. Although increasing the inhalation resistance can help enhance the deposition of drug particles in the lungs, the airflow resistance also increases during both inhalation and exhalation, causing the patient's treatment experience to be negatively affected. Accordingly, the nebulizer and the housing assembly provided by the present disclosure utilize the flexible shielding member that is disposed on the outer end of at least one of the openings to cover or uncover at least one of the openings based on the direction of the airflow entering or exiting the mouthpiece, thereby dynamically adjusting the volume of the airflow passing through the openings.

When the patient inhales through the mouthpiece 13 and draws the aerosol in the air chamber C1 into the body, the airflow within the air chamber C1 flows toward the mouthpiece 13, thereby pulling the flexible shielding member 2 to tightly adhere to the second opening 12. Since the second opening 12 is completely covered by the flexible shielding member 2, and the flexible shielding member 2 is tightly adhered to the second opening 12, the air in the external environment is prevented from entering the air chamber C1 through the second opening 12, thereby increasing the inhalation resistance during the patient's inhalation. In order to smoothly inhale the aerosol (i.e., the nebulized medication), the patient will naturally adjust his breathing rhythm to adopt a slower and deeper inhalation. The slower and deeper inhalation not only facilitates smooth entry of the aerosol into the respiratory tract, but also allows the aerosol to penetrate deeper into the lungs, thereby enabling the drug particles to be effective deposited in the lung tissues and enhancing the therapeutic efficacy.

On the other hand, when the patient exhales through the mouthpiece 13, the airflow flows toward the first openings 11 and the second opening 12 on the housing 1 (i.e., an airflow direction N2 in FIG. 9). Since the flexible shielding member 2 is flexible and sheet-shaped, it lifts when pushed by the airflow, such that the second opening 12 is exposed on the housing 1 and the exhaled air can exit smoothly. In other words, during the patient's exhalation, the flexible shielding member 2 does not cover the second opening 12, effectively reducing the exhalation resistance and ensuring smooth breathing to improve the patient's comfort. In addition, the design of the nebulizer D prevents pressure buildup inside the housing 1 during exhalation, avoiding any negative impact on the nebulization performance in subsequent inhalation, thereby improving the stability and efficiency of the nebulizer D.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A nebulizer, comprising:
a main body;
a housing engaged with the main body, wherein an air chamber is provided inside the housing, the housing includes a mouthpiece, a sidewall of the housing is provided with a plurality of openings, and the plurality of openings are in fluid communication with the mouthpiece via the air chamber;
a flexible shielding member disposed on the sidewall of the housing, wherein the flexible shielding member covers at least one of the openings;
a cup body engaged with the housing, wherein a liquid storage chamber is provided inside the cup body, a bottom of the housing is provided with a through hole that is in fluid communication with the liquid storage chamber, and the liquid storage chamber is in fluid communication with the air chamber via the through hole; and
a nebulizing module disposed in the cup body and located directly above the through hole;
wherein the flexible shielding member is configured to dynamically adjust an airflow passing through the openings based on a direction of the airflow entering or exiting the mouthpiece.

2. The nebulizer according to claim 1, wherein the openings are further divided into two first openings and at least one second opening, the two first openings and the mouthpiece are located on opposite sides of the housing, and the flexible shielding member covers the at least one second opening.

3. The nebulizer according to claim 2, wherein the flexible shielding member covers an outer end of the at least one second opening.

4. The nebulizer according to claim 2, wherein, when a user inhales through the mouthpiece, the airflow in the air chamber flows toward the mouthpiece, and the flexible shielding member is drawn by the airflow to adhere to the at least one second opening.

5. The nebulizer according to claim 2, wherein, when a user exhales into the mouthpiece, the airflow in the air chamber flows toward the openings, and the flexible shielding member is pushed by the airflow and lifted, thereby exposing the at least one second opening.

6. The nebulizer according to claim 2, wherein a projection of the flexible shielding member that is projected onto the sidewall of the housing completely overlaps the at least one second opening.

7. The nebulizer according to claim 2, wherein a quantity of the second opening is one, and the second opening is located between the two first openings.

8. The nebulizer according to claim 7, wherein the flexible shielding member has a vent hole, and the second opening is in fluid communication with the vent hole.

9. The nebulizer according to claim 2, wherein a quantity of the second opening is two, the two second openings are located beneath the mouthpiece, and the two second openings are closer to the mouthpiece than the first opening.

10. The nebulizer according to claim 9, wherein the housing includes a partition wall that is disposed inside the mouthpiece, a hollow space inside the mouthpiece above the partition wall defines the air chamber, another hollow space inside the mouthpiece beneath the partition wall defines a sensing chamber, the air chamber and the sensing chamber are structurally independent and not in fluid communication with each other, and the two second openings are in fluid communication with the sensing chamber.

11. The nebulizer according to claim 1, wherein the flexible shielding member includes at least one fastening member, the sidewall of the housing is provided with at least one fastening hole that corresponds to the fastening member, the flexible shielding member is fixedly connected on the sidewall through the at least one fastening member being fittingly engaged with the at least one fastening hole.

12. A housing assembly, comprising:
a housing having an air chamber inside, wherein the housing includes a mouthpiece, a sidewall of the housing is provided with a plurality of openings, and the plurality of openings are in fluid communication with the mouthpiece via the air chamber; and
a flexible shielding member disposed on the sidewall of the housing, wherein the flexible shielding member covers an outer end of at least one of the openings;
wherein the flexible shielding member is configured to dynamically adjust a volume of an airflow passing through the openings based on a direction of the airflow entering or exiting the mouthpiece.

13. The housing assembly according to claim 12, wherein the openings are further divided into two first openings and at least one second opening, the two first openings and the mouthpiece are located on opposite sides of the housing, and the flexible shielding member covers the at least one second opening.

14. The housing assembly according to claim 13, wherein, when a user inhales through the mouthpiece, the airflow in the air chamber flows toward the mouthpiece, and the flexible shielding member is drawn by the airflow to adhere to the at least one second opening.

15. The housing assembly according to claim 13, wherein, when a user exhales into the mouthpiece, the airflow in the air chamber flows toward the openings, and the flexible shielding member is pushed by the airflow and lifted, thereby exposing the at least one second opening.

16. The housing assembly according to claim 13, wherein a projection of the flexible shielding member that is projected onto the sidewall of the housing completely overlaps the at least one second opening.

17. The housing assembly according to claim 13, wherein a quantity of the second opening is one, and the second opening is located between the two first openings.

18. The housing assembly according to claim 17, wherein the flexible shielding member has a vent hole, and the second opening is in fluid communication with the vent hole.

19. The housing assembly according to claim 13, wherein a quantity of the second opening is two, the two second openings are located beneath the mouthpiece, and the two second openings are closer to the mouthpiece than the first opening.

20. The housing assembly according to claim 19, wherein the housing includes a partition wall that is disposed inside the mouthpiece, a hollow space inside the mouthpiece above the partition wall defines the air chamber, another hollow space inside the mouthpiece beneath the partition wall defines a sensing chamber, the air chamber and the sensing chamber are structurally independent and not in fluid communication with each other, and the two second openings are in fluid communication with the sensing chamber.
